(19) United States
(12) Patent Application Publication
(10) Pub. No.: US 2023/0048693 A1
(43) Pub. Date: Feb. 16, 2023

(54) CANNULA AND OBTURATOR SYSTEM FOR MINIMALLY INVASIVE SURGERY

(71) Applicant: Maruho Medical, Marietta, GA (US)

(72) Inventors: John L. Minck, JR., Reno, NV (US); Brett Andrew Snyder, Reno, NV (US); Ryan Schmid, Reno, NV (US); Brendan D. Thies, Woodstock, GA (US); Justin C. Sluder, Dallas, GA (US)

(73) Assignee: Maruho Medical, Marietta, GA (US)

(21) Appl. No.: 17/876,626

(22) Filed: Jul. 29, 2022

Related U.S. Application Data
(60) Provisional application No. 63/231,833, filed on Aug. 11, 2021.

(51) Int. Cl. A61B 17/34; A61B 17/02

(52) U.S. Cl. CPC A61B 17/3431; A61B 17/0218; A61B 2017/347

(57) ABSTRACT

A cannula for providing a pathway for surgical instruments in a minimally invasive procedure including a flexible body portion having a lumen extending from the proximal region to the distal region, a proximal opening, a distal opening and a flange extending radially outwardly from the distal region and being flexible for insertion through an incision in a body of a patient. A rigid body portion extends proximally of the flexible body portion which is more flexible than the rigid body portion. A first seal is positioned within the rigid body portion and spaced proximally of a region of the flexible body portion distal of the rigid body portion, the seal preventing egress of fluids from the body of the patient.

### BACKGROUND

This application claims priority to provisional application 63/231,833 filed on Aug. 11, 2021, the entire contents of which are incorporated herein by reference.

### TECHNICAL FIELD

This application relates to a system and method for accessing the body for minimally invasive surgery, and more particularly to a cannula, insertable with an obturator, for providing a pathway for surgical instruments.

### BACKGROUND OF RELATED ART

The advantages of minimally invasive surgery over open surgery are well known. These advantages include reduced trauma, lower risk of infection, shorter hospital stays, faster patient recovery time, reduced costs, etc. In minimally invasive surgery, such as endoscopy and arthroscopy, a cannula is inserted through the skin and advanced to the target site. The cannula has a lumen forming a passageway (pathway) for insertion of surgical instruments therethrough to perform the surgical procedure at the target site.

In the design of cannulas, a difficult balance needs to be made between ensuring the cannula does not back out after placement in the patient's body and ensuring any structure to prevent the cannula from backing out does not significantly hinder either advancement or withdrawal of the cannula through the body tissue. That is, on one hand, the cannulas, once positioned, should be designed to ensure they are not displaced, which can occur as instruments are inserted and removed through the cannula. If the cannula is displaced during the surgical procedure, then it is no longer at the desired target site which will adversely affect surgical instrument placement. If the cannula backs out fully from the body, then it needs to be reinserted, adding complications to the surgery as well as increasing the surgery time. On the other hand, such structure to aid retention of the cannula provided on the outer wall which interacts with the tissue to provide resistance to displacement needs to not be overly aggressive such that it would increase difficulty of inserting and removing the cannula or cause trauma to tissue during insertion and/or removal. Cannulas to date have not optimized the balance of these two competing factors.

Additionally, the cannula needs to prevent fluid from escaping from the body prior to and during insertion of the instruments therethrough as well as when the instruments remain in the cannula during the surgical procedure. It would also be beneficial to provide such fluid blocking structure which does not increase the difficultly of insertion of instrumentation through the cannula, and more ideally, would increase the ease of insertion for the clinician. The cannulas also need to be designed to maintain the integrity of the seal during instrument insertion and removal through the cannula.

Several cannulas are disclosed in the prior art such as by way of example the hybrid cannula of U.S. Pat. No. 9,119,663, the arthroscopic cannula of U.S. Pat. No. 9,675,379, the portal device of U.S. Pat. No. 8,128,601, the arthroscopic cannula of U.S. Publication No. 2021/0128142, the portal device of U.S. Pat. No. 8,128,601 and the cannula of U.S. publication 2019/0239922. Each of these devices/cannulas suffers from one or more deficiencies and fails to address the problems enumerated above.

The need therefore exists for an easy to manufacture cannula that provides for ease of insertion, sufficient retention to prevent displacement and slipping out of the incision and effective sealing to prevent egress of body fluids through the cannula.

### SUMMARY OF THE INVENTION

The present invention overcomes the problems and deficiencies of the prior art. The present invention provides in one aspect a cannula providing a pathway to the target site for various surgical instruments. The present invention provides in another aspect an obturator and cannula system to ease penetration through the patient's skin and advancement to the surgical site followed by removal of the obturator to provide a pathway for instrumentation to the target tissue through the cannula lumen. These components and system are discussed in more detail below.

The devices, systems and methods of the present invention advantageously provide an easy to manufacture cannula that provides for ease of insertion, sufficient retention to prevent displacement and slipping out of the incision and effective sealing to prevent egress of body fluids through the cannula prior to and during insertion/withdrawal of surgical instruments through the cannula. These advantages will be appreciated by the detailed description of preferred embodiments below. The devices, systems and methods of the present invention can be used in various endoscopic and arthroscopic procedures, including for example, shoulder, hip, knee, elbow, etc.

The present invention also provides a manufacturing method for the cannula of the present invention in which flexible and rigid portions are attached.

In accordance with one aspect of the present invention, a cannula for providing a pathway for surgical instruments in a minimally invasive procedure is provided, the cannula comprising a flexible body portion having a longitudinal axis, a proximal opening, a distal opening and a lumen extending along the longitudinal axis from the proximal region to the distal region. A flange extends radially outwardly from the distal region of the flexible body portion and is flexible for insertion through an incision in a body of a patient. A rigid body portion extends proximally of the flexible body portion, and the flexible body portion is composed of a more flexible material than the rigid body portion. A first seal preventing egress of fluids from the body of the patient is positioned within the rigid body portion and is spaced proximally from the region of the flexible body portion which is distal of the rigid body portion.

In some embodiments, the flange on the flexible body portion includes a plurality of fingers with spaces therebetween. In some embodiments, the fingers are foldable backward (proximally) during insertion and return to a transverse position with respect to the longitudinal axis of the body portion when placed within the body of the patient. In some embodiments, the flange is configured to provide a propeller like motion when the cannula is manually rotated by a clinician during insertion. In some embodiments, the flange has a star shaped configuration, (also considered an octopus arm shape), although other shapes are also contemplated.

The flexible body portion of the cannula can include a plurality of tissue capturing features extending radially from an outer wall of the intermediate region. In some embodiments, the plurality of tissue capturing features comprise a plurality of spaced apart projecting surfaces having a distal face and a proximal face, wherein the distal face has a first angle and the proximal face has a second different angle. In preferred embodiments, the first angle of the distal face is less than the second angle of the proximal face to provide a shallower angular surface on a lead in side. In some embodiments, the flexible body portion is tapered in a distal direction.

In some embodiments the cannula is formed by overmolding the flexible body portion to the rigid body portion.

In accordance with another aspect of the present invention, a cannula for providing a pathway for surgical instruments in a minimally invasive procedure is provided. The cannula comprises an elongated body, a longitudinal axis and a lumen extending from the proximal region to the distal region and having a proximal and distal opening. A flange is positioned at the distal region of the elongated body and is flexible for insertion through an incision in a body of a patient. The flange includes a plurality of fingers spaced apart to form a gap between adjacent fingers and the plurality of fingers are configured to provide a propeller like motion when the cannula is manually rotated by a clinician during insertion into the body of the patient. A first seal is positioned within the body, the seal preventing egress of fluids from the body of the patient;

In some embodiments of the cannulas disclosed herein, the first seal comprises a valve configured to prevent egress of fluids when an instrument is not present in the lumen of the cannula. In some embodiments, the cannula further comprises a second seal including a valve positioned proximal of the first seal to prevent egress of fluids when a surgical instrument is passed through the second seal. In some embodiments, an end cap at a proximal end of the body is provided wherein the first and second seals are sandwiched between the end cap and an internal wall of the cannula.

The foregoing cannulas in some embodiments are part of a system which includes in combination an obturator. Thus, in another aspect, the present invention provides the cannula in combination with an obturator having a distal portion including a slot, e.g., an L-shaped slot, having a radial portion and an axial portion, and the cannula has a projection extending from an internal wall to engage the slot of the obturator. In preferred embodiments, the obturator and cannula have a first rotational position wherein the projection engages the axial portion of the slot and a second rotational position wherein the projection engages the radial portion of the slot to secure the obturator in positon within the cannula.

In accordance with another aspect, the present invention provides a method of manufacturing a cannula comprising:
a) providing a rigid body composed of a first rigid material;
b) overmolding a flexible body portion made of a second flexible material and having a lumen extending therein;
c) positioning a seal within the rigid body spaced proximally of a region of the flexible body portion which is distal of the rigid body; and
d) attaching an end cap to the cannula to sandwich the seal between the end cap and an internal wall of the rigid body to maintain the seal within the rigid body.

In some embodiments the method further comprises the step of positioning a second seal within the rigid body spaced proximally of the lumen in the flexible body portion prior to the step of attaching the end cap.

In accordance with another aspect of the present invention, a method of providing a pathway to a target site during minimally invasive surgery is provided comprising the steps of:
a) inserting an obturator in a lumen of a cannula such that a first region of a slot in an outer wall of the obturator is aligned with an inwardly projecting surface of the cannula;
b) rotating the obturator so that the projecting surface in an internal wall of the cannula engages a second region of the slot;
c) manually rotating the obturator and cannula as a unit such that a plurality of fingers of a flange of the cannula provide a spinning propeller like motion of the obturator and cannula as they are advanced together through the skin of a patient and to the target site;
d) removing the obturator from the cannula; and
e) inserting a surgical instrument through the lumen of the cannula to the target site.

In some embodiments, the plurality of fingers bend back proximally during insertion of the flange through the skin. In some embodiments, the cannula has a flexible body portion and a proximal rigid body portion and a seal within the rigid body portion to prevent egress of fluids.

### BRIEF DESCRIPTION OF THE DRAWINGS

Preferred embodiment(s) of the present disclosure are described herein with reference to the drawings wherein:

FIG. 1 is a perspective view of the cannula in accordance with an embodiment of the present invention;

FIG. 2 is a perspective view of the obturator in accordance with an embodiment of the present invention;

FIG. 3 is an exploded view of the cannula of FIG. 1;

FIG. 4 is a side perspective view of the rigid body portion of the cannula of FIG. 1;

FIG. 5A is a perspective view of the cannula seal of the cannula of FIG. 1;

FIG. 5B is a perspective view of the instrument seal of the cannula of FIG. 1 shown from a distal side;

FIG. 5C is a perspective view of the instrument seal of the cannula of FIG. 1 shown from a proximal side;

FIG. 6 is longitudinal cross-sectional view of the cannula of FIG. 1 with the obturator of FIG. 2 positioned therein;

FIG. 7 is a side view of the cannula of FIG. 1 with the obturator of FIG. 2 positioned therein;

FIG. 8 is a side perspective view of the cannula of FIG. 1 with the obturator of FIG. 2 positioned therein in a non-interlocking position;

FIG. 9 is a view similar to FIG. 8 showing the obturator rotated to a locking position;

FIG. 10 is a front view showing the obturator in the position of FIG. 8;

FIG. 11 is a front view showing the obturator in the position of FIG. 9;

FIGS. 12-16 illustrate a method of insertion of the cannula and obturator of FIGS. 1 and 2 in accordance with an embodiment of the present invention wherein:

FIG. 12 illustrates initial placement of the obturator and cannula against the skin for penetration;

FIG. 13 shows initial penetration of the cannula wherein the fingers of the flange are folded back and the cannula and obturator are rotated and advanced through the tissue;

FIG. 14 shows the cannula and obturator in position in the body cavity;

FIG. 15 shows the obturator being withdrawn from the cannula and the cannula remaining in the body; and

FIG. 16 shows a surgical instrument inserted through the cannula positioned in the body.

### DETAILED DESCRIPTION OF PREFERRED EMBODIMENTS

The present invention provides a cannula that provides a pathway for surgical instrumentation in endoscopic or arthroscopic surgery. The cannula of the present invention has features that achieve one or more of the following: 1) facilitates ease of insertion into the body; 2) reduces the chances of cannula movement or slippage out of the body once in position within the body; 3) provides an optimal balance of flexibility and rigidity; 4) enhances sealing of the cannula prior to insertion of an instrument through the cannula; 5) enhances sealing of the cannula while an instrument is positioned in the cannula; and 6) facilitates one-handed insertion and manipulation. Features of the cannula that achieve these advantages are described in detail below. It should be understood that the various features (#1-#6) of the cannula can achieve one or more of the foregoing and that the cannula need not include all these features but can include any number of features to achieve any number of the foregoing advantages.

The present invention can also include a system that comprises the cannula and an obturator to aid insertion. The obturator is inserted into the cannula and interlocks with the cannula for insertion together as a unit. This cannula/obturator system is described in more detail below.

The present invention can further include a method of manufacturing the cannula which secures flexible and rigid portions and protects the cannula sealing system. The manufacturing method is described in more detail below.

Note as used herein, the term “distal” refers to the region or portion further from the user and the term “proximal” refers to the region or portion closer to the user. As used herein, the term “substantially” or “approximately” or “about” or “generally” means+25% of a given value or measurement or feature.

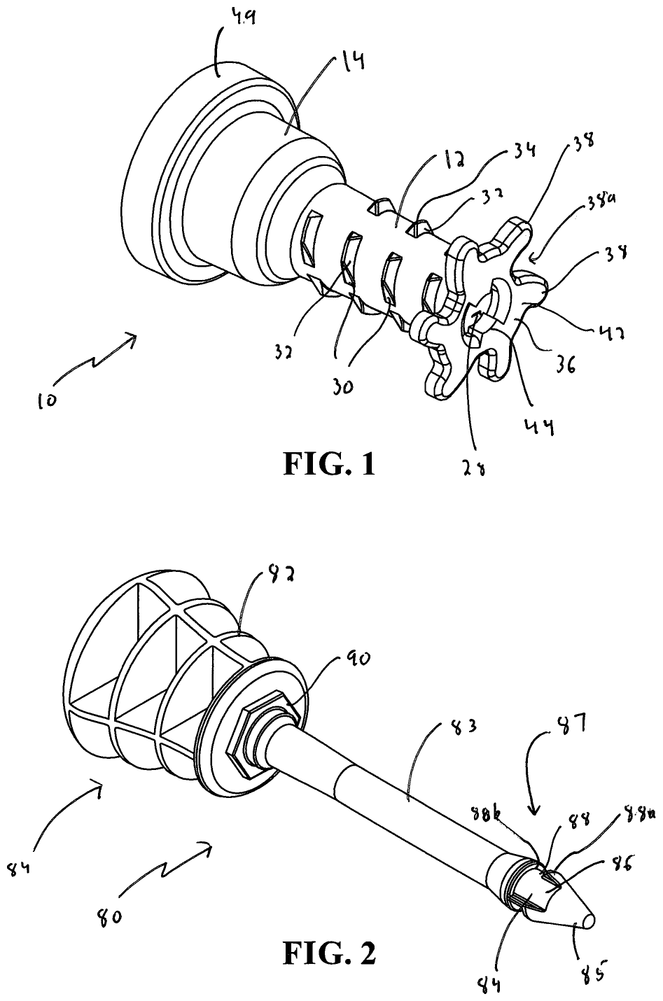

Referring now to the drawings and particular embodiments of the present disclosure, wherein like reference numerals identify similar structural features of the devices disclosed herein, the cannula of the present invention will be discussed first. Within initial reference to FIGS. 1 and 3, the cannula is designated generally by reference numeral 10 and includes a flexible body 12, a rigid body 14, a seal mechanism 16 and a proximal end cap 18. The rigid body 14 is positioned proximal of the flexible body 12 and contains the seal mechanism 16 therein. The flexible body 12 is also referred to herein as the flexible body portion 12 and the rigid body 12 is also referred to herein as the rigid body portion 12.

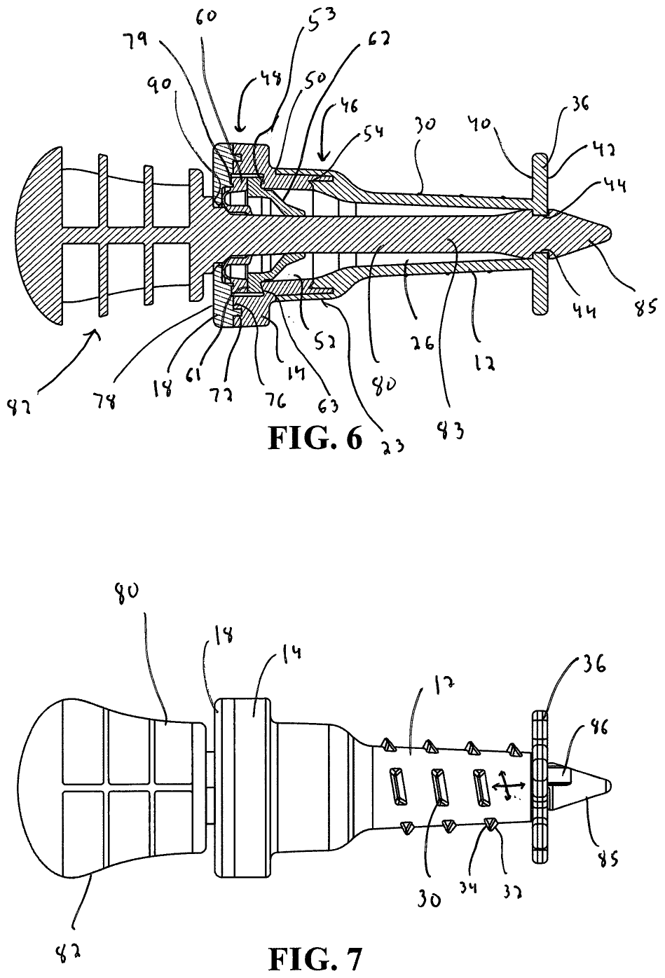

Flexible body 12 has a proximal portion 20, a distal portion 22 and an intermediate portion 24 extending between the proximal and distal portions 20, 22. In the illustrated embodiment, the flexible body 12 has a taper in a distal direction so the diameter is reduced in a distal direction to aid insertion through tissue. The taper can also assist sealing the cannula 10 against the surgical incision and help prevent cannula pistoning. Thus, the distal end of the cannula (the neck) tapers from the proximal end of the neck to the distal end of the neck to prevent the cannula from pistoning into the body as well as helping to get a good seal between the outside of the cannula neck and the skin incision. However, in alternate embodiments, the taper could be in a proximal direction or the flexible body 12 can be of uniform diameter. It is also contemplated that the outer diameter of the flexible body 12 can change via a stepped construction rather than a taper. The flexible body 12 also has an enlarged diameter region 23 at the proximal portion. Flexible body 12 has a lumen 26 (see FIG. 6) extending therethrough with a proximal opening for entry of surgical instrumentation and a distal opening 28 for exit of surgical instrumentation. The lumen 26 is also dimensioned and configured to receive obturator 80 therethrough for insertion of the cannula 10 as discussed below. The flexible body 12 can be formed of various polymeric materials such as TPE (thermoplastic elastomer) or silicone by way of example. As shown, the flexible body 12 is preferably circular in cross-section, although other cross-sectional configurations are contemplated, e.g., oval.

On the outer surface of the wall of the flexible body 12, along the intermediate region 24, are a plurality of tissue engagement features 30. The tissue engagement features 30, also referred to herein as tissue engagement members 30 or tissue capture members, extend radially outwardly from the outer surface (outer wall) and are spaced apart radially and axially as shown. In preferred embodiments, the engagement members 30 occupy only a fraction of the radial circumference of the flexible body 12, as shown for example in FIG. 1, however, in alternate embodiments, the engagement members 30 can occupy a greater or smaller area than shown. Stated another way, the engagement members 30 can have a length and/or width larger or smaller than that shown in the Figures. Additionally, the height (radial distance from the outer surface of the wall of the flexible body 12) of the engagement members 30 can be different than that shown.

In the illustrated embodiment, there are four longitudinally extending rows of engagement features, with three rows having three engagement members and one row having four engagement members. The engagement members of each row are substantially radially aligned with an engagement member of an adjacent row as shown. However, it should be appreciated that number of engagement members in each row can be greater or less than three, the number of longitudinal rows can be greater or less than the four shown and the axial spacing can be different than that shown and can be equidistantly or non-equidistantly spaced. Also, the engagement members in each row can be non-axially aligned with the engagement members of the adjacent row.

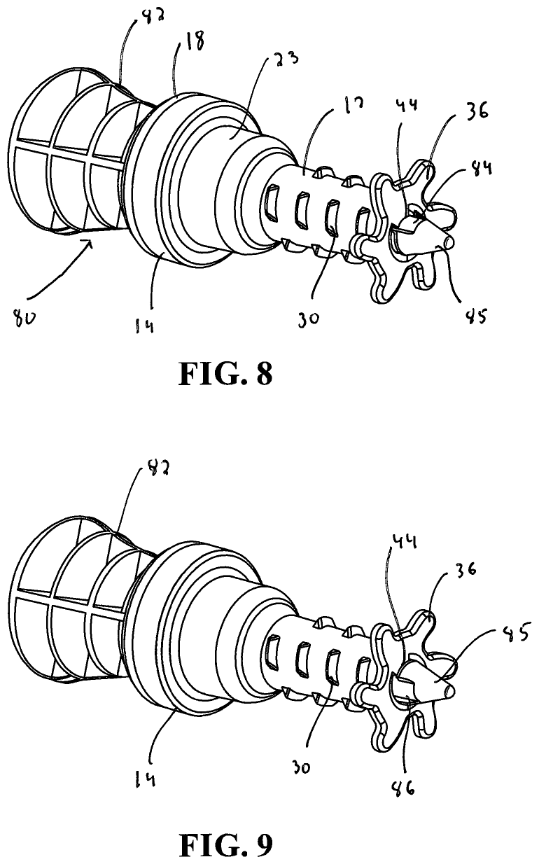

With reference to the preferred embodiment of FIGS. 1 and 3, each engagement member 30 is formed at a slight angle to a longitudinal axis of the flexible body 12 (see e.g., FIG. 7) such that a longitudinal axis along the length of the member 30 would be at an acute or obtuse angle to the longitudinal axis of the flexible body 12 (see arrows of FIG. 7). Note the engagement members 30 can be formed integrally (monolithically) with the flexible body 12 in accordance with the manufacturing method discussed below, e.g. formed by injection molding of the flexible body 12.

Each engagement member 30 has a distal face 32 (facing distally toward the distal end of the flexible body 12) and a proximal face 34 (facing proximally toward the proximal end of the flexible body 12). As shown, the distal face 32 has an angle (slope) less than the angle of the proximal face 34 as it provides a lead in angle for insertion. The proximal face 30 has a greater angle as it functions to limit retraction of the cannula 10. By way of example, the steeper angle of the proximal face 34 could be between about 70 degrees and about 80 degrees and the shallower angle of the distal face 32 could be between about 40 degrees and 45 degrees. It should be appreciated that other angles of the proximal face 34 and distal face 32 are also contemplated. Thus, the engagement members 30 are configured and angled to limit the chances of the cannula 10 retracting or pistoning during use, e.g., during insertion and withdrawal of instrumentation therethrough, while not adversely affecting insertion of the cannula 10 through tissue into the body cavity, i.e., without increasing difficulty of insertion which can cause trauma to tissue. The tissue engagement members 30 also help insertion as the cannula 10 is rotated into tissue. It should be appreciated that other shapes and sizes of the engagement features 30 are also contemplated which can achieve the functions/benefits described herein.

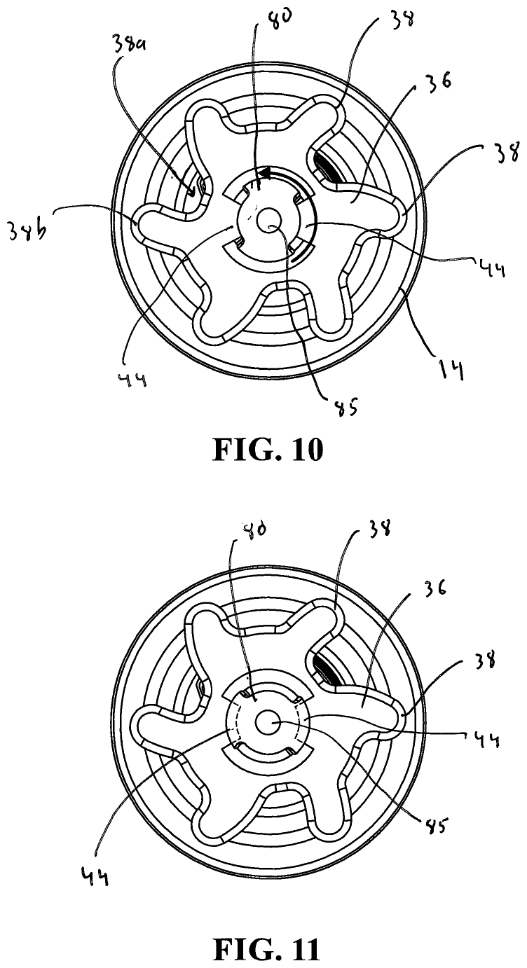

At the distal opening 28 of flexible body 12 are a pair of projections or tabs 44 projecting inwardly toward the longitudinal axis of the flexible body 12 (see FIGS. 6 and 10). The projections 44 are configured and dimensioned to engage slots in the obturator 80 in the manner discussed below. In the illustrated embodiment, two obturator-engaging projections 44 are provided, spaced 180 degrees apart, although a different number and/or different spacing are also contemplated.

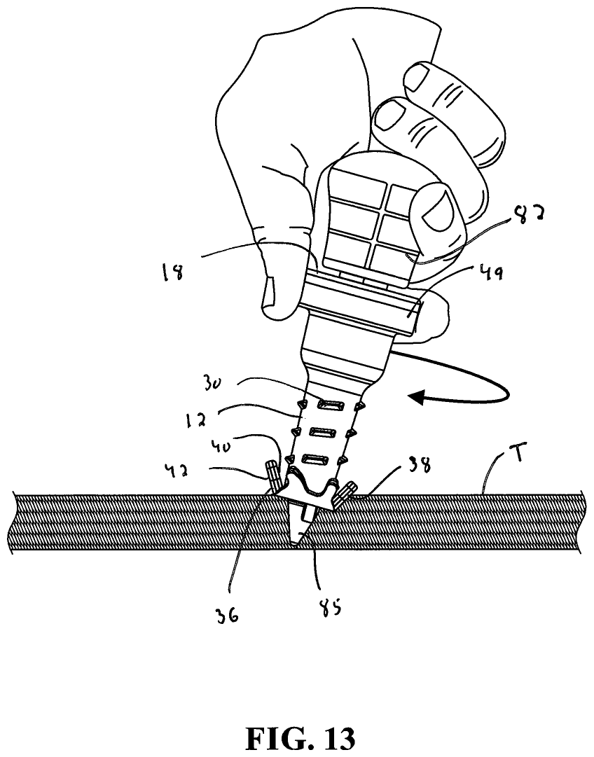

Flexible body 12 has a flange 36 at a distal end extending radially outwardly thereby increasing the diameter of the flexible body portion 12. The flange 36 surrounds the distal opening 28 and includes a plurality of spaced apart fingers 38 with gaps 38a between the fingers 36. The flange 36 therefore has a star-shape with the individual fingers terminating in curved surfaces 38b (FIGS. 10 and 11). Six fingers 38 (and thus six gaps 38a) are shown in the Figures, although a different number of fingers and different shape or size of the fingers are also contemplated as long as it achieves its functions as described herein. The flange 36 performs two functions. One function is to provide a stop for retraction of the cannula 10. A second function is to aid insertion as the fingers 38 bend back proximally during insertion and form a propeller like device as the cannula 10 is rotated during insertion. This proximal bending of the fingers 38 wherein the fingers 38 bend back on themselves is illustrated in FIG. 13 and discussed below in conjunction with the discussion of the method of use/insertion.

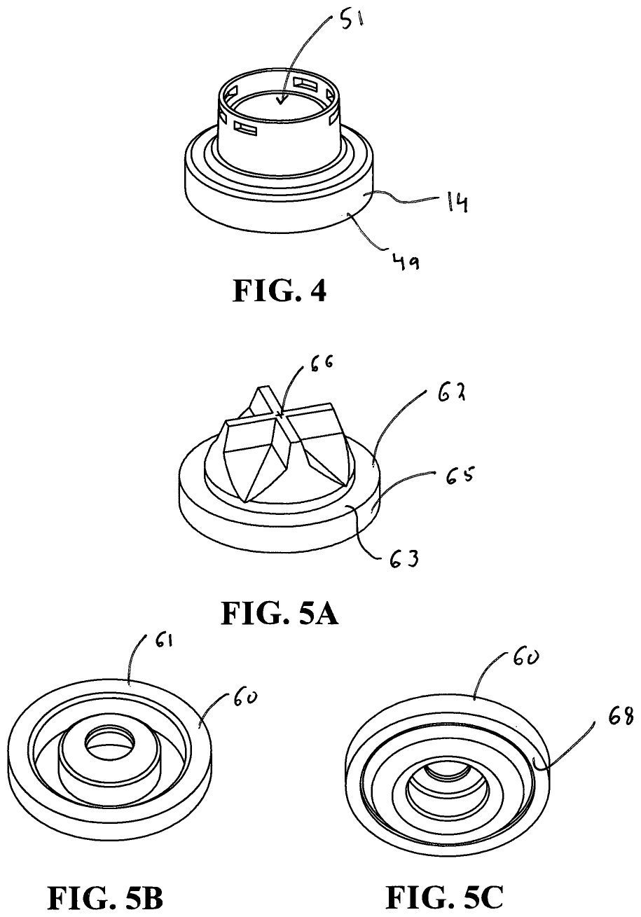

As mentioned above, cannula 10 includes a rigid body 14 at a proximal end of the flexible body 12. With reference to FIGS. 1, 3, 4 and 6, rigid body 14 has a distal region 46 and a proximal region 48 forming a larger diameter region 49. A shoulder 50 abuts the proximal edge of the flexible body 12. Underlying region 54 forms a shoulder 54 engaging an internal wall of underlies enlarged diameter region 23 of the flexible body 12. A lumen 52 extends through the rigid body 14, aligned and in communication with lumen 26 of flexible body 12. Internal shoulder 53 of the rigid body engages (abuts) the distal seal 62 of the seal mechanism 16. As shown in the cross-sectional view of FIG. 6, shoulder 54 is within the outer flexible body 12; shoulder 53 is outside the confines of the flexible body 12 and proximal thereto. The rigid body 14 can be formed of polycarbonate or other rigid materials. The rigid body 14 also gives the user a rigid component to hold onto during insertion and removal of instruments as the back end is not flexible to provide a better grip and prevent or limit deformation of the cannula.

Circumferential slot 56 (FIG. 3) at a proximal end of rigid body 14 is dimensioned to receive circular projection 72 of end cap 18 (FIG. 6). End cap 18 further has a more inward (radially) circular projection 79 extending distally therefrom to engage the proximal seal 60 described below. End cap 18 can be made of polycarbonate or other rigid materials. End cap 18 has an opening 74 aligned with opening 51 in rigid body 14 and aligned with the lumens 52 and 26 of the rigid body 14 and flexible body 12, respectively. The outer (proximal) surface of end cap 18 is designated by reference numeral 78 and the inner (distal) surface, which contacts the proximal surface of rigid body 14, is designated by reference numeral 76.

Turning to the seal mechanism 16 and with reference to FIGS. 1, 5A, 5B and 6, the seal mechanism includes a distal seal 62 and a proximal seal 60, both preferably composed of elastomeric materials such as silicone. The seals (valves) 60, 62 can in some embodiments be transparent or opaque. The distal seal 62 provides a cannula seal which seals the cannula from egress of fluid in the absence of an instrument inserted or positioned in the lumen of the cannula, i.e., in lumens 52 and 26 of the rigid and flexible body portions 14, 12, respectively. The cannula seal (access valve) 62 can be in the form of a slit valve having slit 66 for passage of instruments while remaining closed when no instrument is passed therethrough. The valve thus seals the cannula and enables instruments to be inserted through the slit 66. The proximal seal 60 is an instrument seal which seals the cannula from egress of fluid when an instrument, e.g., obturator, grasper, cutting instrument, etc. is inserted through or positioned within the lumen of the cannula 10, i.e., lumen 52 and 26 of the rigid and flexible body portions 14, 12, respectively.

With reference to FIG. 6, both seals 62 and 64 are positioned within the rigid body 14 and rigidly fixed therein. As shown, surface 63 of flange (rim) 65 of seal 62 abuts shoulder 53 of rigid body 14. This engagement is within the rigid body 14 and proximal of the region of the flexible body portion 12 which is distal of the rigid body 14, and is fully within the confines of the rigid body 14. Proximal seal 60 includes an elastic membrane with a slot or opening to open when an instrument is inserted therethrough and seals around the instrument. The proximal surface 68 of proximal seal 60 abuts the distal surface 76 of end cap 18. The distal surface 61 of proximal seal 60 abuts the proximal surface of distal seal 62. This engagement is within the rigid body 14 and is spaced proximally from the flexible body 12 and is outside the confines of the flexible body 12. As shown, the entire proximal seal 60 is spaced proximally from the flexible body 12. The placement/positioning of the seals 60, 62 within the non-compliant rigid body portion 14 prevents bending of the seals during flexing or compression of the flexible body 12 during use. Such bending of the seals could cause leakage. Such bending could also cause closing of the seals. The seals 60, 62 are sandwiched between the end cap 18 and shoulder 53 of rigid body 14 as distal surface 76 of end cap 18 engages proximal surface 68 of proximal seal 60 (with projection 79 of end cap 18 engaging a slot in the proximal surface 68) to apply a distal force on the seals 60, 62 and the distal surface 63 of flange (rim) 65 of distal seal 62 is pressed against internal shoulder 53 of rigid body portion 14.

The method of manufacture will now be discussed. The flexible body portion 12 (formed by injection molding) is overmolded onto the rigid body 14 (see overlapping regions of FIG. 6). Valves 60 and 62 are then placed in the rigid body 14 with the rim 65 of valve 62 engaging shoulder 53 of rigid body 14 (see FIG. 6). The end cap 18 is then fastened to rigid body 14 with circular projection 72 engaging slot 56 (FIG. 3) of the proximal surface of the rigid body portion 14 and circular projection 79 engaging the slot in the proximal seal 60. The end cap 18 can be fastened by ultrasonic welding, glue or other methods. The seals (valves) 60 and 62 are thereby sandwiched between the end cap 18 and the internal shoulder 53 of rigid body 14 to retain the seals within the rigid body 14. It should be appreciated that in alternate embodiments, the rigid body 14 and flexible body 12 can be attached by other methods.

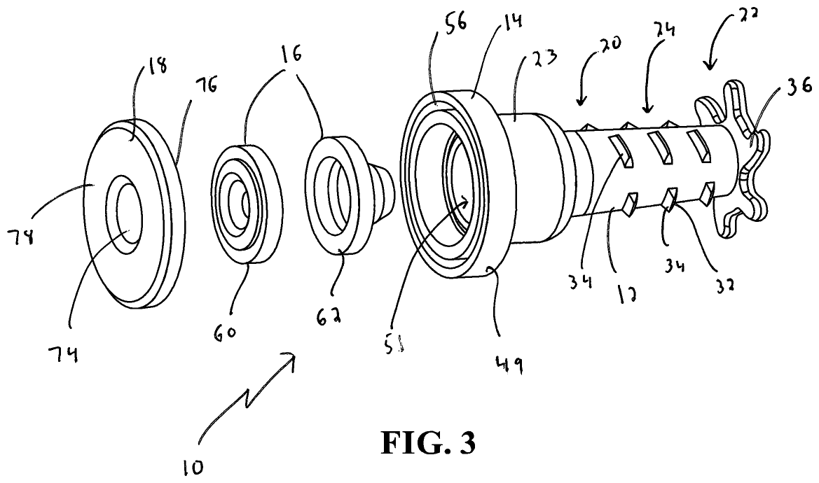

The cannula 10 can be inserted with the aid of obturator 80 removably positioned therein. With reference to FIG. 2, obturator 80 has a body portion 83, a distal portion 87 and a proximal portion 82 forming a grip portion for the user/clinician. The obturator 80 terminates at distal portion 87 in a conical tip 85, although other shaped tips to aid penetration through body tissue are also contemplated.

The obturator 80 has at the distal portion 87 two cannula receiving L-shaped slots 84 for engagement with the obturator engaging projections (tabs) 44 of the flexible body portion 12 of cannula 10. Each slot 84 has a radial region 88 transverse to a longitudinal axis of the obturator (e.g., 90 degrees to the longitudinal axis, although other angles are also contemplated). Each slot 84 also has a longitudinal (axial) region 86 aligned, or substantially aligned, with the longitudinal axis of the obturator 80. Axial region 86 and radial region 88 are connected. The slots 84 are preferably spaced apart 180 degrees. Different spacing and a different number of slots are also contemplated.

When the obturator. 80 is positioned within lumen 26 of cannula 12, projections 44 of cannula 12 engage with the axial region 86 or the radial region 88 of each slot 84, depending on the rotational position of the obturator 80 (or relative rotational positions of the obturator 80 and cannula 12). That is, on insertion, the projections (tabs) 44 of the flexible body portion 12 ride up the axial slot regions 86 of slots 84 until the obturator 70 at distal surface (shoulder) 90 contacts the proximal face 78 of cap 18 (This contact is shown in FIG. 6). At this depth of insertion, the forward (distal) position of obturator 80 within cannula 12 is indicated. A tactile indicator can be provided to indicate to the user this position. In this forward position, tabs 44 are aligned with the radial slot 88.

To lock (interlock) the obturator 80 with the cannula 10, the obturator 80 is rotated 90 degrees so each tab 44 of flexible body portion 12 engages a radial slot 88. In this interlocking position, distal wall 88a and proximal wall 88b of radial slot 88 prevent respectively distal and proximal movement of the obturator 80 within the cannula 10. This interlocking aids one handed insertion as described below. In the illustrated embodiment, the obturator 80 is rotated 90 degrees clockwise to engage the radial slot 88; in alternate embodiments, the obturator can be rotated counterclockwise 90 degrees to engage the radial slot. With other slot configurations, a different degree of rotation can be utilized. To remove the obturator 80, the obturator 80 is rotated in the reverse direction so each tab 44 disengages the radial slot 88 and re-engages the axial slot 86 and the obturator 80 can then be removed. This is discussed below in conjunction with the method of use/insertion of the system of the present invention. Note the obturator 80 is described as rotating with respect to the cannula for the interlocking, however, it is also contemplated that the cannula can be rotated with respect to the obturator 80 or both the obturator 80 and cannula 10 are rotated in opposing directions to achieve the foregoing tab-slot engagement/interlocking.

As shown in the cross-sectional view of FIG. 6, the obturator 80 includes a shoulder 90 which engages the proximal surface 78 of end cap 18 to limit its distal insertion into the rigid body portion 14 and flexible body portion 12 of cannula 10. The body portion 83 of obturator can be tapered or alternatively of uniform outer diameter along its length as shown. Note the body portion 83 of obturator 80 is preferably solid, although in alternate embodiments it can have a lumen extending therein. The obturator 80 is composed of a rigid material which resists flexing to provide sufficient force during insertion. In the illustrated embodiment, the obturator has a larger diameter region in the region of the slots 86, 88.

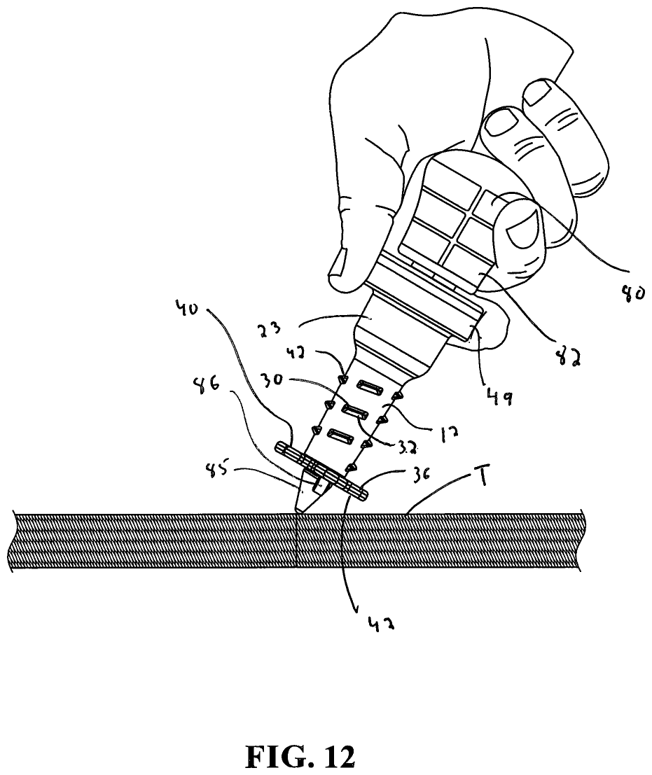

Turning now to the method of use of the system of the present invention, and with reference to FIGS. 12-16, in the first step the obturator 80 is inserted into the cannula 10 (through seals 60 and 62) so that the conical tip 85 extends beyond the flange 36 of flexible body portion 14 as shown in FIG. 12. Thus, the tip 85 is exposed to aid insertion. In this position, the shoulder 90 abuts the proximal face 78 of end cap 18 (FIG. 6) to restrict further distal advancement of the obturator 80. In this position, the tabs 44 of the flexible body portion 12 of cannula 12 are in the axial slot regions 86 of obturator 80 adjacent radial regions 88.

Once inserted into the cannula 10, the obturator 80 is rotated as shown in FIG. 13 so that each obturator engaging projection (tab) 44 of the flexible body portion 12 engages the radial region 88 of slot 84 of obturator 80 to lock/interlock the obturator 80 and cannula 10 (see FIGS. 10 and 11). In this position, distal and proximal movement of the obturator 80 within cannula 10 is restricted (by radial slot walls 88a, 88b) so that the clinician, as shown in FIG. 13, can grip the grip portion 82 of obturator 80 and the enlarged region 49 of rigid body portion 14 of cannula 12. As can be appreciated, the system of the present invention allows for control, e.g., insertion, with one hand. This one handed operation can be seen in FIG. 13 where the user's palm grips the obturator 80 at the gripping portion 82 and the user's finger tips, e.g. the thumb and little finger, grip the enlarged region 49 of the rigid body portion 14 of the cannula 10.

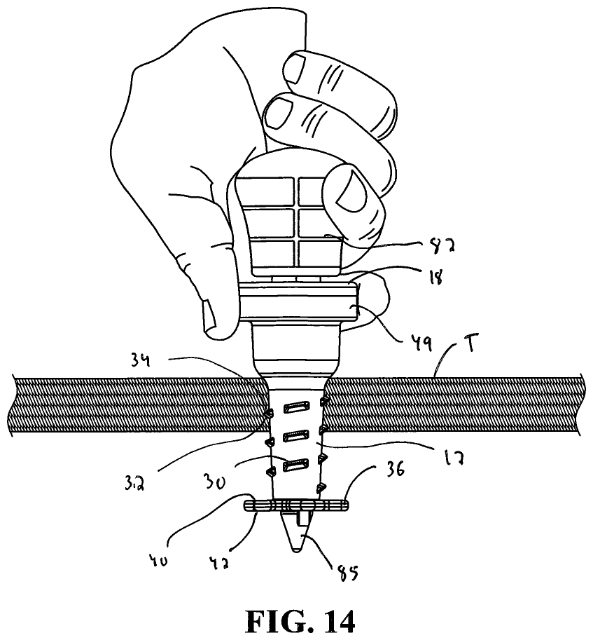

As the cannula 10 is being inserted through the incision and through tissue T, the fingers 38 of flange 36 bend backward (proximally) from the transverse position of FIG. 12 to the position of FIG. 13. In this position/condition, as the cannula 10 is rotated through tissue T during insertion, the fingers 38 of flange 36 rotate (spin) in a propeller-like motion to aid insertion (movement in a distal direction) into the body cavity. The insertion position wherein the cannula 10, with the assistance of obturator 80, is positioned in the body cavity adjacent the target site is shown in FIG. 14.

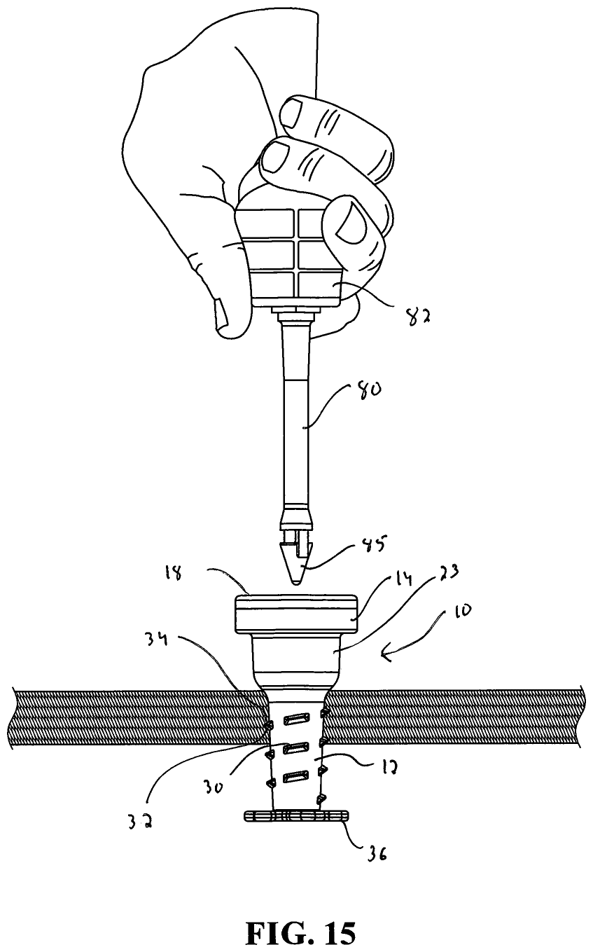

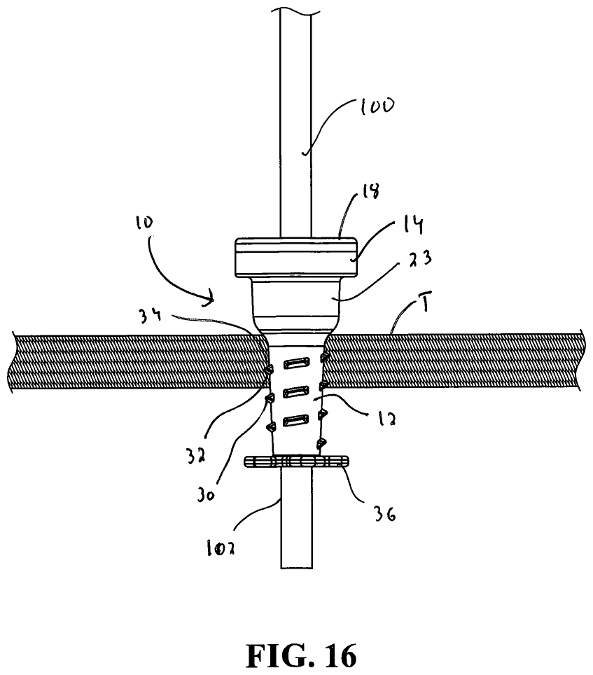

Once the cannula 10 is positioned adjacent the target site, the obturator 80 is rotated in the reverse direction to disengage from tabs 44 and then removed from the lumen 26 of the flexible body portion as shown in FIG. 15. Instruments such as instrument 100 can then be inserted through the seals 60, 62 and through the lumen 26 with the distal end 102 of the instrument 100 exiting past the distal end of the flexible body portion 12 for treating the target site (see FIG. 16). Note the tissue engagement members 30 retain the flexible body portion 12 in position. Several of the tissue engagement members 30 are shown engaged, however, it should be appreciated that additional members 30 could be engaged depending on the thickness of tissue or the number of engagement members 30 in the longitudinal row(s). Further note that seal 60 seals around the obturator 80 and subsequent instruments as described above, with seal 62 sealing the cannula when an obturator or instrument is not positioned in the cannula 10. After the surgical procedure, the cannula 10 is rotated out of the tissue T for removal from the patient's body.

It should be appreciated, that in alternate methods of use, the cannula 10 can be inserted without an obturator.

Although the apparatus and methods of the subject invention have been described with respect to preferred embodiments, those skilled in the art will readily appreciate that changes and modifications may be made thereto without departing from the spirit and scope of the present invention as defined by the appended claims. Persons skilled in the art will understand that the various embodiments of the disclosure described herein and shown in the accompanying figures constitute non-limiting examples, and that additional components and features may be added to any of the embodiments discussed herein without departing from the scope of the present invention.

It will be understood by those skilled in the art that the above particular embodiments are shown and described by way of illustration only. The principles and the features of the present disclosure may be employed in various and numerous embodiments thereof without departing from the scope and spirit of the disclosure as claimed. The above-described embodiments illustrate the scope of the disclosure but do not restrict the scope of the disclosure.

Additionally, persons skilled in the art will understand that the elements and features shown or described in connection with one embodiment may be combined with those of another embodiment without departing from the scope of the present invention and will appreciate further features and advantages of the presently disclosed subject matter based on the description provided.

Throughout the present invention, terms such as “approximately,” “about”, “generally,” “substantially,” and the like should be understood to allow for variations in any numerical range or concept with which they are associated. It is intended that the use of terms such as “approximately”, “about”, “substantially”, and “generally” should be understood to encompass variations on the order of 25%, or to allow for manufacturing tolerances and/or deviations in design.

The recitation of numerical ranges by endpoints includes all numbers within the range.

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present invention.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

What is claimed is:

1. A cannula for providing a pathway for surgical instruments in a minimally invasive procedure, the cannula comprising:
a) a flexible body portion having a proximal region, a distal region, and an intermediate region extending between the proximal region and distal region, the flexible body portion further having a flange, a longitudinal axis, a lumen extending along the longitudinal axis from the proximal region to the distal region, a proximal opening at the proximal region and a distal opening at the distal region, the flange extending radially outwardly from the distal region of the flexible body portion and being flexible for insertion through an incision in a body of a patient;
b) a rigid body portion extending proximally of the flexible body portion, the flexible body portion being more flexible than the rigid body portion; and
c) a first seal positioned within the rigid body portion and spaced proximally of a region of the flexible body portion distal of the rigid body portion, the seal preventing egress of fluids from the body of the patient.

2. The cannula of claim 1, wherein the flange includes a plurality of fingers with spaces therebetween forming a star shape.

3. The cannula of claim 2, wherein the fingers are foldable back during insertion and return to a transverse position with respect to the longitudinal axis when placed within the body of the patient.

4. The cannula of claim 3, wherein the flange is configured to provide a propeller like motion when the cannula is manually rotated by a clinician during insertion.

5. The cannula of claim 1, further comprising a plurality of tissue capturing members extending radially from an outer wall of the intermediate region of the flexible body portion.

6. The cannula of claim 5, wherein the plurality of tissue capturing members comprise a plurality of spaced apart projecting surfaces, the projecting surfaces having a distal face and a proximal face, wherein the distal face has a first angle and the proximal face has a second different angle.

7. The cannula of claim 6, wherein the first angle of the proximal face is greater than the first angle of the distal face to provide a shallower angular surface on a lead in side.

8. The cannula of claim 1, wherein the first seal comprises a first valve configured to prevent egress of fluids when an instrument is not present in the lumen of the cannula.

9. The cannula of claim 8, further comprising a second seal comprising a valve positioned proximal of the first seal to prevent egress of fluids when a surgical instrument is passed through the second seal, the second seal positioned proximal of the flexible body portion.

10. The cannula of claim 9, wherein the first and second seals are composed of elastomeric materials and are fixed within the rigid body portion such that the first and second seals are not bent during flexing or compression of the cannula.

11. The cannula of claim 10, further comprising a proximal end cap, the first and second seals sandwiched between the end cap and an internal wall of the rigid body portion.

12. The cannula of claim 1, wherein the flexible body portion has a taper in a distal direction to keep the cannula from pistoning into the body of the patient and help provide a seal between an outside of the cannula and the incision.

13. The cannula of claim 1 in combination with an obturator, the obturator having a proximal portion and a distal portion, the distal portion having a slot having a radial portion and an axial portion, and the cannula having a projection extending from an internal wall to engage the slot of the obturator.

14. The cannula of claim 13, wherein the obturator and cannula have a first rotational position wherein the projection engages the axial portion of the slot and a second rotational position wherein the projection engages the radial portion of the slot to secure the obturator in position within the cannula.

15. The cannula of claim 12, wherein the flexible body portion is composed of a more flexible material than the rigid body portion.

16. A cannula for providing a pathway for surgical instruments in a minimally invasive procedure, the cannula comprising:
a) a proximal region, a distal region, and an elongated intermediate region extending between the proximal region and distal region;
b) a longitudinal axis and a lumen extending from the proximal region to the distal region along the longitudinal axis and having a proximal opening and a distal opening;
c) a flange at the distal region, the flange flexible for insertion through an incision in a body of a patient, the flange including a plurality of fingers spaced apart to form a gap between adjacent fingers and the plurality of fingers are configured to provide a propeller like motion when the cannula is manually rotated by a clinician during insertion into the body of the patient; and
d) a first seal positioned within the body, the first seal preventing egress of fluids from the body of the patient.

17. The cannula of claim 16, wherein the first seal comprises a first valve configured to prevent egress of fluids when an instrument is not present in the lumen of the cannula and the cannula further comprises a second seal positioned proximal of the first seal to prevent egress of fluids when a surgical instrument is passed through the second seal.

18. The cannula of claim 16, in combination with an obturator, the obturator having a proximal portion and a distal portion, the distal portion including a slot having a radial portion and an axial portion, and the cannula having a projection extending from an internal wall thereof to engage the slot of the obturator.

19. A method of manufacturing a cannula comprising:
a) providing a rigid body composed of a first rigid material;
b) overmolding a flexible body portion made of a second flexible material and having a lumen extending therein;
c) positioning a seal within the rigid body spaced proximally of a region of the flexible body portion spaced distally of the rigid body; and
d) attaching an end cap to the cannula to sandwich the seal between the end cap and an internal wall of the rigid body to maintain the seal within the rigid body portion.

20. The method of claim 19, further comprising the step of positioning a second seal within the rigid body spaced proximally of the flexible body portion prior to the step of attaching the end cap.
